(19) United States
(12) Patent Application Publication
(10) Pub. No.: US 2021/0353952 A1
(43) Pub. Date: Nov. 18, 2021

(54) ANGLED LASER THERAPY HANDPIECE

(71) Applicant: Bruce Coren, West Palm Beach, FL (US)

(72) Inventor: Bruce Coren, West Palm Beach, FL (US)

(21) Appl. No.: 16/875,876

(22) Filed: May 15, 2020

(51) Int. Cl. A61N 5/06

(52) U.S. Cl. CPC A61N 5/06; A61N 2005/067

(57) ABSTRACT

A medical laser for use in medical healing applications is provided. The medical laser comprises a hand piece having a main body containing an optical sub-assembly for delivering a dose of radiation. The hand piece also has a hand-held grip angularly disposed from said main body, wherein said handheld grip has a vertical centerline that is approximately perpendicular to a horizontal centerline of the main body. The medical laser also comprises a high-powered class IV laser generator source in optical communication with said optical sub-assembly via a fiber optic cable. The medical laser also comprises a docking station separate from and not connected to the laser generator, the docking station providing two shaped profiles accommodating two shaped profiles of the handheld grip.

### BACKGROUND OF THE INVENTION

### 1. Field of the Invention

The present invention relates to a system and method for delivering laser therapy and, more particularly, to an improved ergonomic laser therapy hand piece device and method for the delivery of high dose laser therapy.

### 2. Description of the Related Art

Medical lasers are used in a variety of medical healing applications. A deep penetrating action is used in a variety of medical applications to relieve pain and speed up recovery from injury. Such treatments are known to offer a less invasive alternative to more traditional medical practices such as injections and surgery. In such treatments, photons from a laser light source are directed to penetrate the skin and underlying tissues where they are absorbed by targeted cells and converted into energy. The result is that cell membrane permeability is altered with a host of cellular results including, inter alia:
1. Stimulation of ATP;
2. Stimulation of respiratory chain;
3. Increased DNA and RNA synthesis;
4. Increased levels of beta endorphins and serotonin; and
5. Enhanced Collagen synthesis.

When provided in conjunction with a high-powered class IV laser, an alternative treatment is accomplished as an alternative to cortisone injection and, in some cases, surgeries.

Some examples of the types of injuries that such laser therapy may be provided for with human patients include:
Cervical strain/sprain;
Headaches;
Tennis/golfers elbow;
Overuse/repetitive stress injuries;
Carpal tunnel syndrome;
De Quervains Tenosynovitis;
Osteoarthritis;
ITB syndrome;
Bursitis;
Rotator cuff strain;
Bicep tendonitis;
Postural strain overuse;
Failed surgical back syndrome;
Spinal stenosis;
Disk bulge herniation;
Jumpers knee;
Ligament/Meniscal injuries;
Neuropathy (diabetic and other forms);
Heel pain; Plantar fasciitis; Achilles tendonitis;
Arthritis (degenerative joint disease); Nerve pain; Non-healing wounds; Pain and healing following surgery;
Trigger points and muscle spasms; and ulcerations and open wounds.

Similarly, laser therapy may also be used an alternative treatment for equivalent conditions for use with animals. Some examples of the types of injuries that such laser therapy may be provide for with animal patients include, inter alia, arthritis/degenerative joint disease; tendon suspensory and ligament disorders; back and muscle disorders; disk and neurological pathologies; stifle hip and sacriilliac disorders; epyphysitis, carpitis, sesamoiditis; laminitis and navicular disease; pre and post-surgical hard and soft tissue trauma; wound healing—acute or chronic; dental conditions—surgical and non-surgical; otitis externa; and non-specific dermatological conditions.

It is preferable that in the application of such laser therapy treatments a laser therapy device is used that provides an end effector having an ergonomic configuration in order to provide for improved application, decreased stress injuries to the physician or technician, or provide for alleviation of the risks, inefficiencies or discomforts associated with conventional therapeutic laser hand pieces. Consequently, a need has been felt for providing a device to achieve such objectives.

### SUMMARY OF THE INVENTION

It is a general object of the present invention to provide a means to alleviate these, and other problems associated with the current state of the art of laser therapy devices. It is a more specific object of the present invention to achieve the foregoing by teaching an improved ergonomic laser therapy hand piece device and method for the delivery of high dose laser therapy.

It is a feature of the present invention to provide such a laser therapy hand piece that includes an angular or pistol-type hand grip. Briefly described according to a preferred embodiment of the present invention, a laser therapy device improved is provided having an end effector configuration with an angled configuration grip, similar to what is seen in a pistol grip.

The front of the laser hand piece enclosure houses an optic subassembly, that in turn houses a lens and fiber adapter. A fiber cable exits at the rear of the Laser Hand Piece, where the fiber cable terminates with a SMA905 connector and a B series Lemo connector. The SMA connector couples to the TMA Diowave class 4 laser's, laser diode and the series B Limo connector connects to the Lemo K series connection on a diowave laser. The hand piece has a switch situated on the front of the handle which is used to activate/deactivate the laser using the index finger.

Additionally, a docking station may be provided to secure the hand piece when not in use. It is an advantage of the present invention to provide a laser therapy end effector having a hand piece configuration that is supported, used or repositioned by a specialist such that the user's grip is repositioned to a more comfortable one. Such an advantage relieves a user from the tight, wrapped grip currently necessary for properly manipulating a laser therapy wand.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a front perspective view showing the complete laser hand piece and docking station design with its cable according to the preferred embodiment of the present invention;

FIG. 2 is a front perspective view showing the complete laser hand piece with the cable removed;

FIG. 3 is a right-side elevational view showing the complete laser hand piece with the cable removed;

FIG. 4 is a front elevational view showing the complete laser hand piece with the cable removed;

FIG. 5 is a rear elevational view showing the complete laser hand piece with the cable removed;

FIG. 6 is a top plan view showing the complete laser hand piece with the cable removed;

FIG. 7 is sectional view taken along line A-A of FIG. 6;

FIG. 8 is an exploded front perspective view thereof;

FIG. 9 is an exploded back perspective view thereof;

FIG. 10 is a front perspective view showing the rear housing element of the laser hand piece 10 for use in conjunction with the preferred embodiment of the present invention;

FIG. 11 is a rear perspective view thereof;

FIG. 12 is a rear elevational view thereof;

FIG. 13 is a front elevational view thereof;

FIG. 14 is a right-side elevational view thereof, the left side elevational view being a mirror image;

FIG. 15 is top plan view thereof;

FIG. 16 is a bottom plan view thereof;

FIG. 17 is a front perspective view showing the front housing element of the laser hand piece for use in conjunction with the preferred embodiment of the present invention;

FIG. 18 is a rear perspective view thereof;

FIG. 19 is a front elevational view thereof;

FIG. 20 is rear elevational view thereof;

FIG. 21 is a right side elevational view thereof, the left side elevational view being a mirror image;

FIG. 22 is a top plan view of thereof;

FIG. 23 is a bottom plan view thereof;

FIG. 24 is a rear perspective view of a docking station 70 for use in conjunction with the laser hand piece 10 of the preferred embodiment of the present invention;

FIG. 25 is a front perspective view thereof;

FIG. 26 is a top plan view thereof;

FIG. 27 is a bottom plan view thereof;

FIG. 28 is a right side elevational view thereof, the left side elevational view being a mirror image;

FIG. 29 is a rear elevational view thereof;

FIG. 30 is a front elevational view thereof;

FIG. 31 is a front perspective view showing the optical subassembly 20 for use with the laser hand piece 10 in accordance with the preferred embodiment of the present invention;

FIG. 32 is a rear perspective view thereof;

FIG. 33 is a front exploded perspective view thereof;

FIG. 34 is a rear perspective view of the subassembly housing 52 for use in the optical subassembly 20 of FIG. 31 through FIG. 33;

FIG. 35 is a front perspective view thereof;

FIG. 36 is a top plan view thereof;

FIG. 37 is a right side elevational view thereof;

FIG. 38 is a front elevational view thereof;

FIG. 39 is a rear elevational view thereof;

FIG. 40 is a front perspective view of the lens cap 56 for use in the optical subassembly 20 of FIG. 31 through FIG. 33;

FIG. 41 is a rear perspective view thereof;

FIG. 42 is a top plan view thereof;

FIG. 43 is cross sectional view taken along line B-B of FIG. 42;

FIG. 44 is a front elevational view thereof;

FIG. 45 is a rear elevational view thereof;

FIG. 46 is an front side perspective view;

FIG. 47 is a rear side perspective view;

FIG. 48 is a top view;

FIG. 49 is a cross sectional view taken along line C-C of FIG. 48; and

FIG. 50 is a front view.

### DESCRIPTION OF THE PREFERRED EMBODIMENTS

The best mode for carrying out the invention is presented in terms of its preferred embodiment, herein depicted within the Figures. It should be understood that the legal scope of the description is defined by the words of the claims set forth at the end of this patent and that the detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

It should also be understood that, unless a term is expressly defined in this patent there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term by limited, by implication or otherwise, to that single meaning. Finally, unless a claim element is defined by reciting the word “means” and a function without the recital of any structure, it is not intended that the scope of any claim element be interpreted based on the application of 35 U.S.C. § 112, sixth paragraph.

1. Detailed Description of the Figures

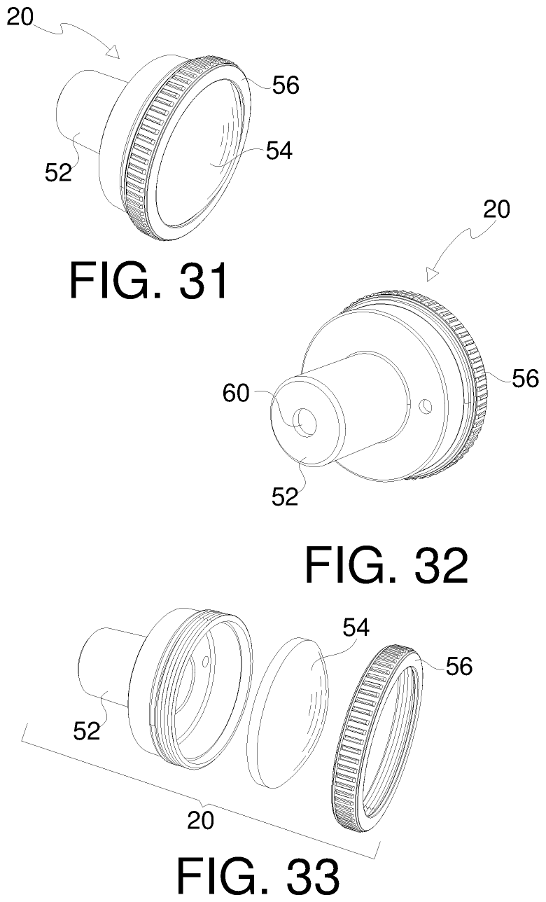

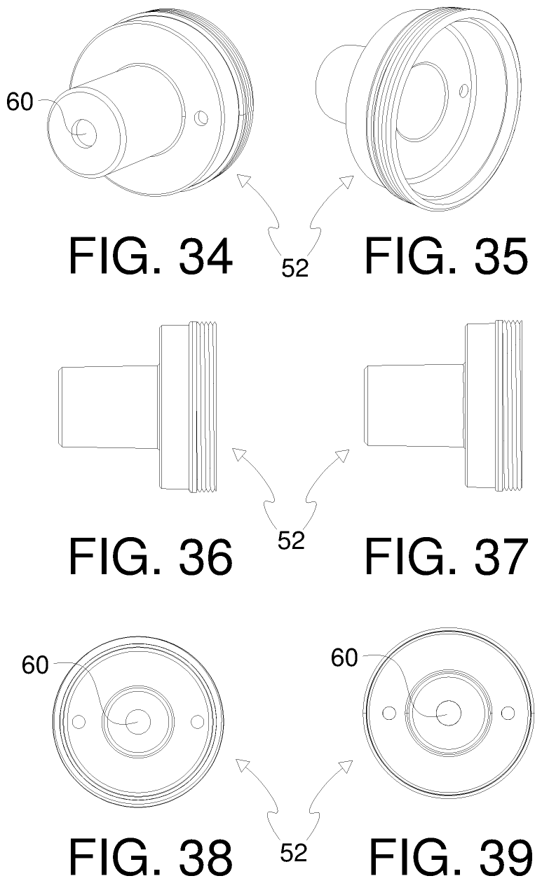

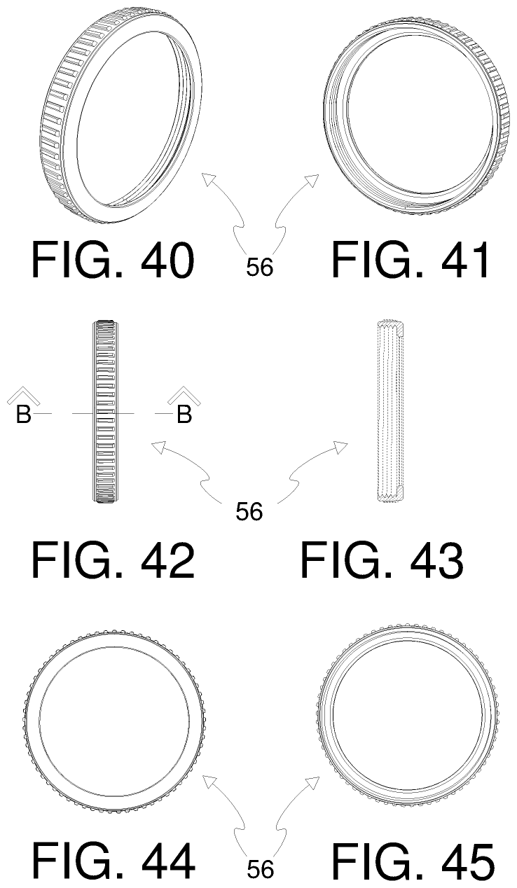

Referring now to the drawings, wherein like reference numerals indicate the same parts throughout the several views, an improved laser therapy hand piece, generally noted as 10, is shown according to the preferred embodiment of the present invention. The hand piece 10 forms an upper head 12 and a lower handle 14 and comprises a housing formed of a front housing element 16 and a rear housing element18. The upper head 12 contains an optical sub-assembly 20, as will be described in greater detail below in conjunction with FIG. 31-50, that connects the hand piece 10 to a laser generator (not shown) by a fiberoptic cable 22. In a preferred embodiment a laser generator may be a High Power—High Dose Laser Therapy (HPLT-HDLP) Class IV laser system such as a Diowave™ Laser System (e.g. ALT Laser, Model VTR75) provided by Technological Medical Advancements, Inc. of West Palm Beach, Fla. or its equivalent.

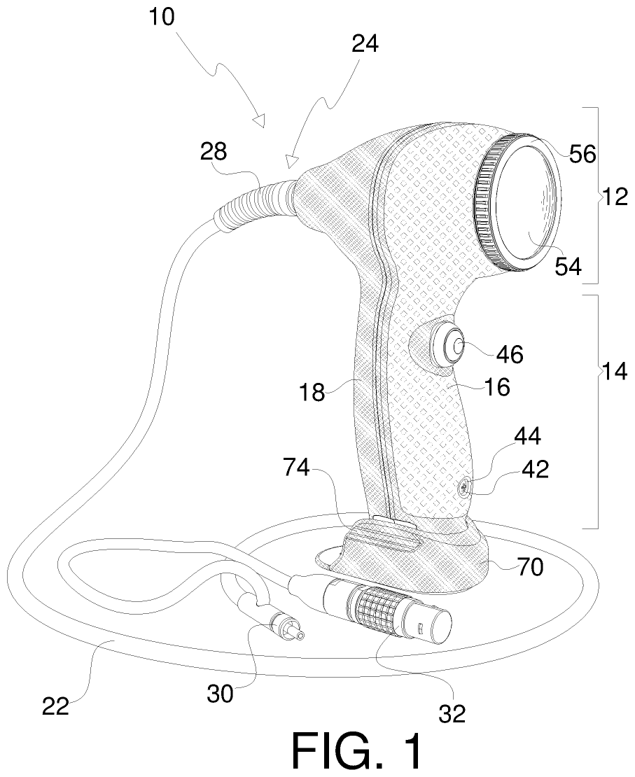

As shown best in conjunction with FIG. 1, the head piece 10 is connected by a fiber optic cable 22 to the laser generator (not shown). The distal end of the fiber optic cable 22 terminates to the rear housing element 18, and eventually to the optical sub assembly 20, as will be described in greater detail below. A strain relief assembly 24 connects the cable 22 to the rear housing element 18 with a central hub 26, which attaches the flexible strain relief member 28 with a locking nut 27. The proximal end of the fiberoptic cable 22 terminates with an optical coupling 30 and an electrical coupling 32. The optical coupling 30 may include an SMA905 connector to couple to the laser generator (not shown) 20 and its laser diode. The electrical coupling 32 may include a Lemo B-Series connector and connects with a Lemo K-series connector at the laser generator.

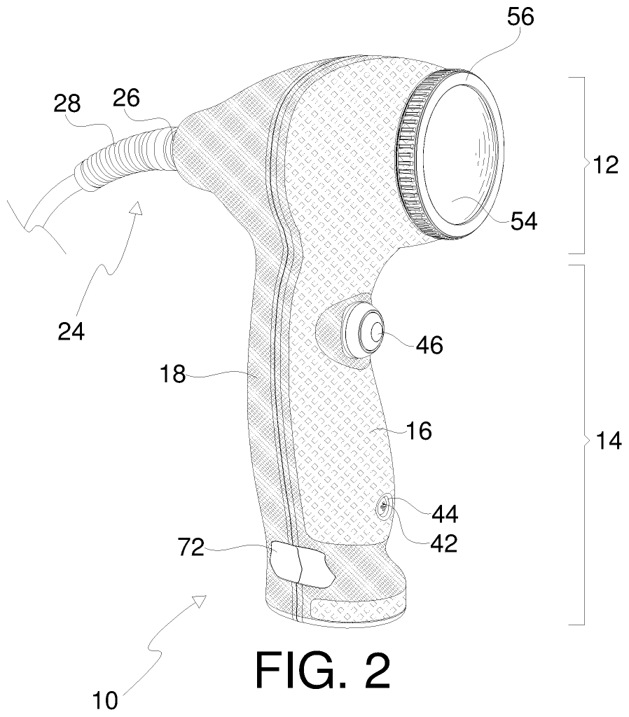

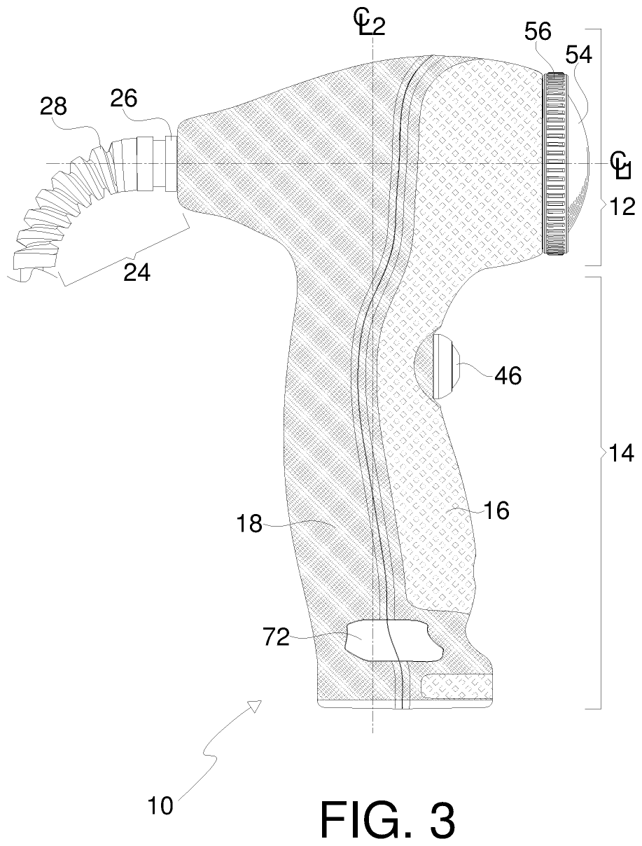

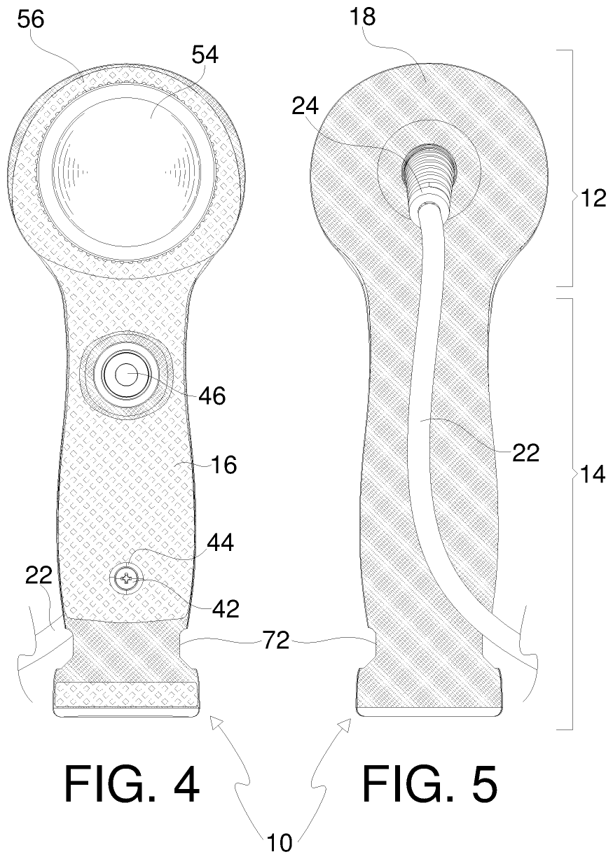

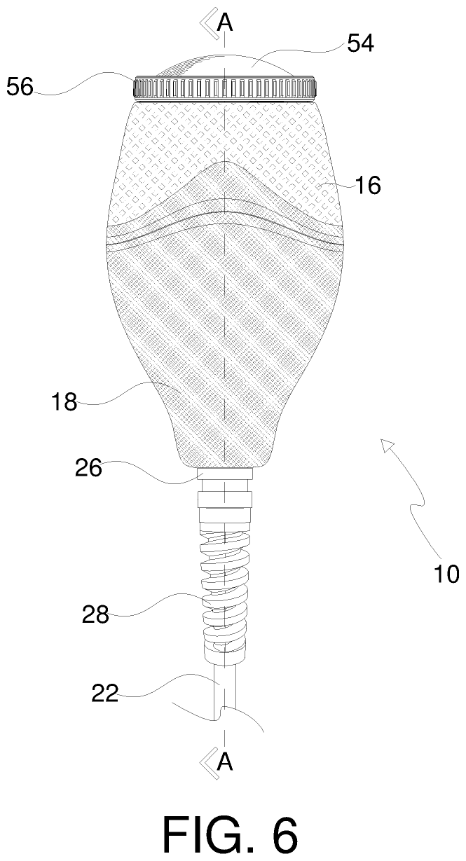

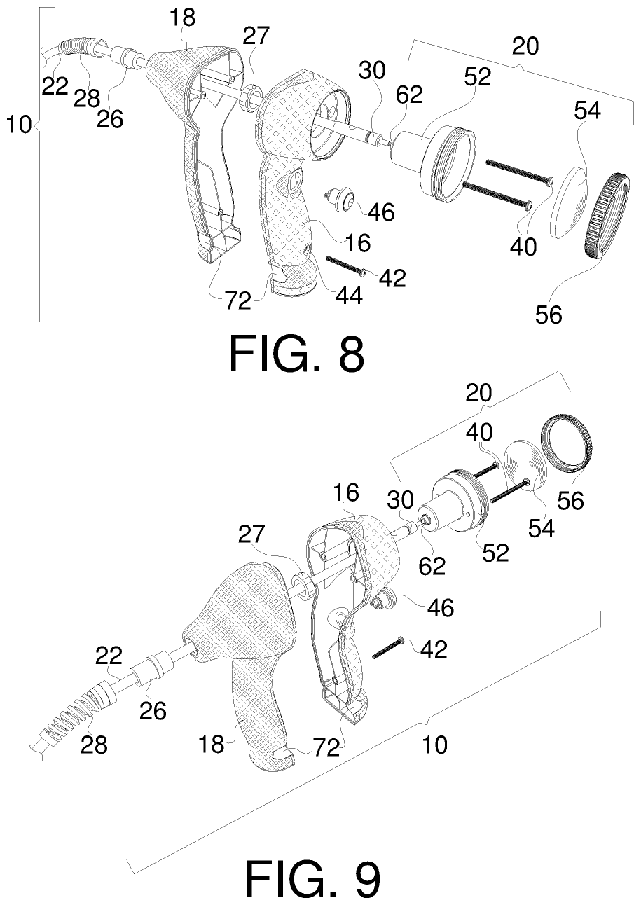

As shown best in conjunction with FIG. 2 through FIG. 9, the hand piece is shown in greater detail. The hand piece 10 forms the head 12 that has a generally horizontal centerline “CL1”. The handle 14 has a generally vertical centerline “CL2”. According to one aspect of the present invention the horizontal centerline “CL1” is angularly disposed relative to the vertical centerline “CL2”.

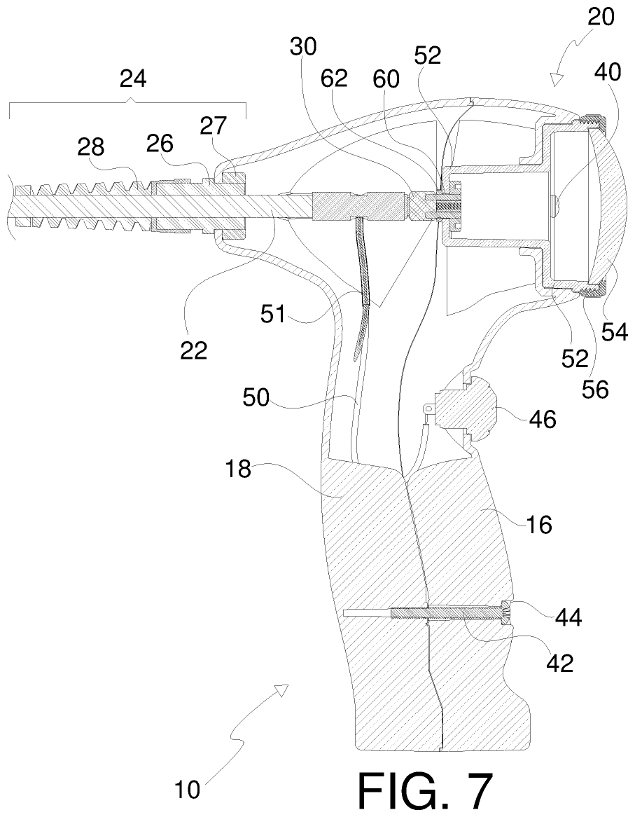

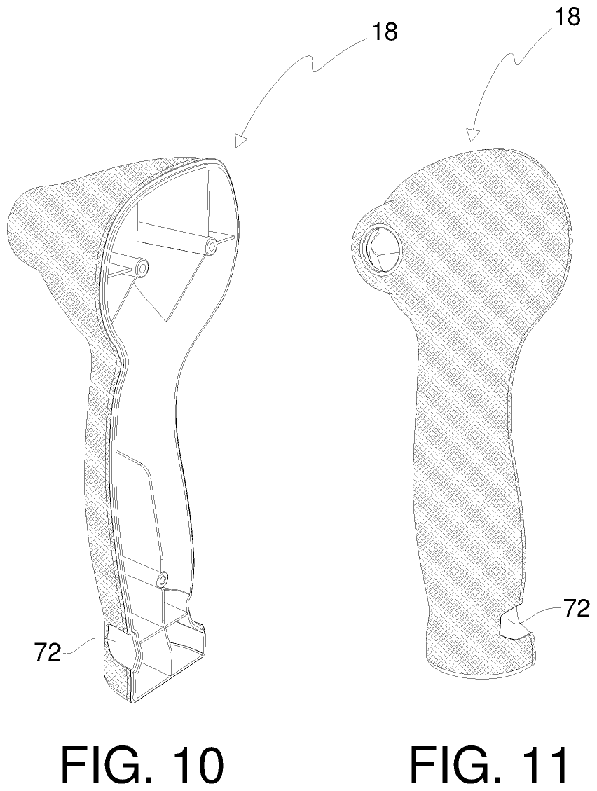

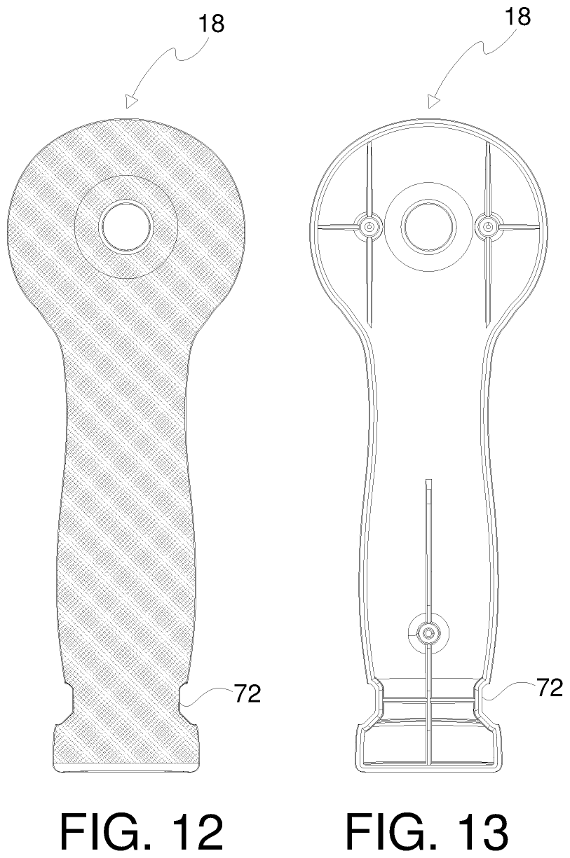

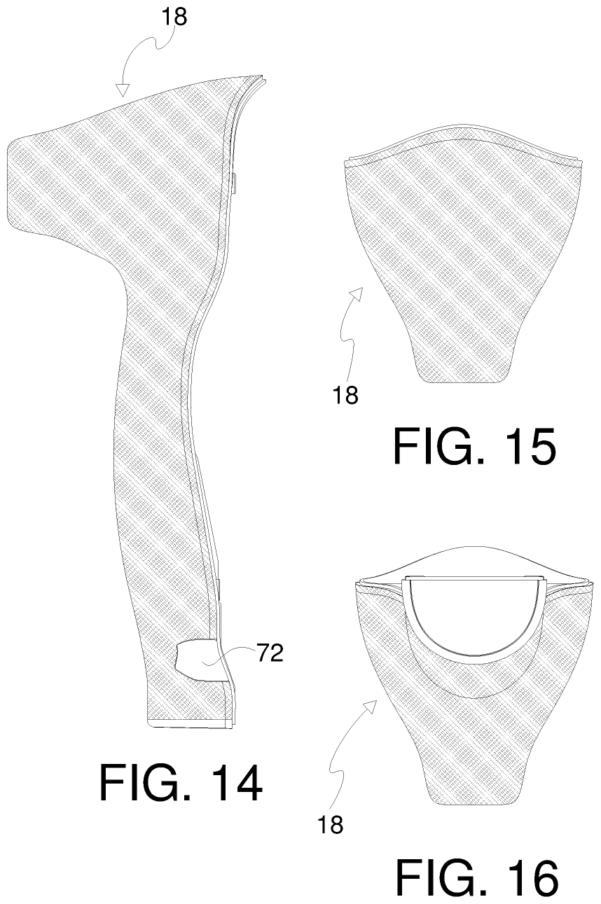

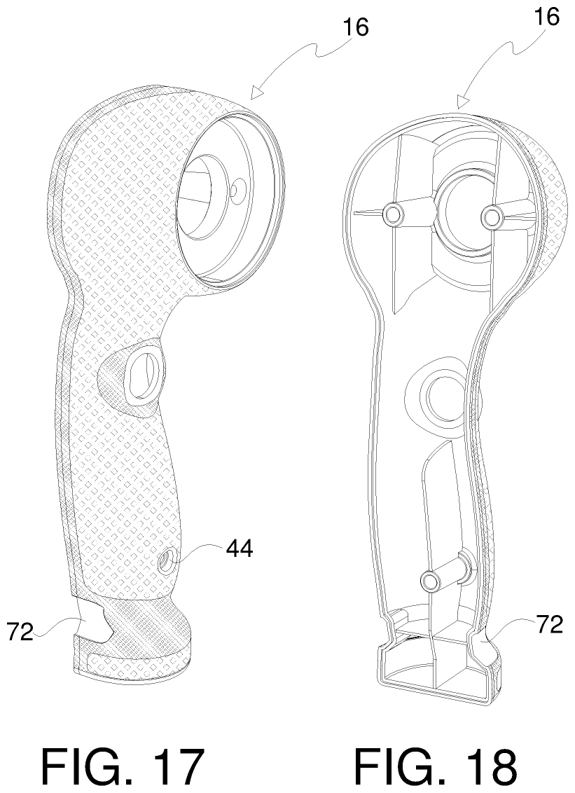

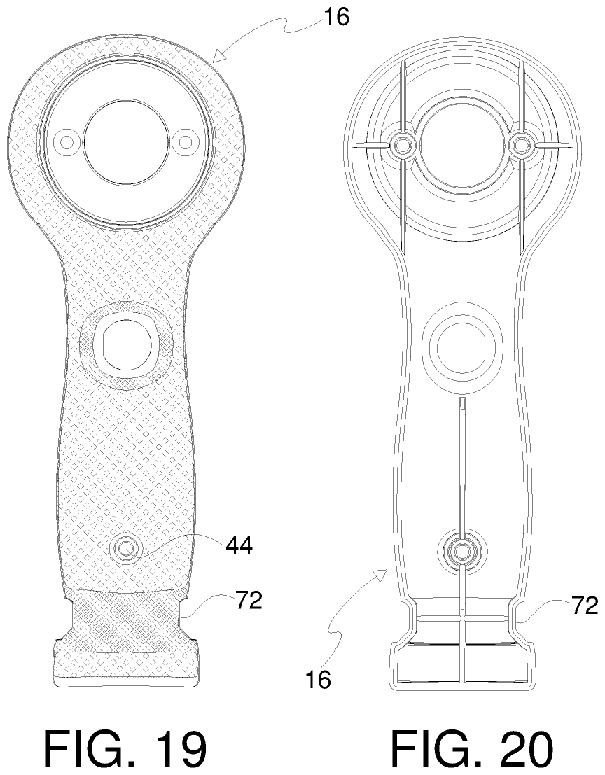

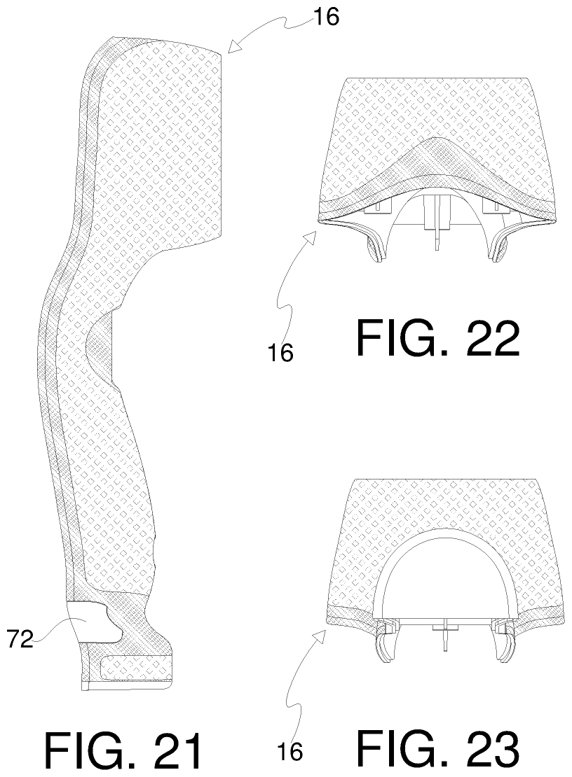

According to yet another aspect of the present invention the two centerlines “CL1” and “CL2” are generally approximately perpendicularly disposed to one another. The head and handle assembly form an enclosure consisting of two parts: the rear housing element 18; and the front housing element 16. The rear housing element 18 is shown in greater detail in conjunction with FIG. 10 through FIG. 16. The front housing element 16 is shown in greater detail in conjunction with FIG. 17 through FIG. 23. These two housing elements 16, 18 mate together and are fastened with fasteners, shown herein as a pair of upper housing fasteners 40 and a lower housing fastener 42. As shown in the current configuration, the upper housing fasteners 40 further secure the optical subassembly 20 within the head 12, and the lower housing fastener 42 secures to the rear housing element 18 through a fastener orifice 44 formed in the front housing element 16. An actuator push button switch 46 is supported by and positioned within the handle 14. As best shown in conjunction with FIG. 7, the switch 46 is in electrical communication with the laser generator (not shown) through electrical wiring 50, 51. The electrical wiring 50, 51 are intended to run in tandem with the fiberoptic cable 22 and, preferably, in a single jacket traveling the length of the cable 22. The cable terminates with an SMA905 connector 30 for the fiber optic cable and a Lemo B series connector 32 for the switch.

As shown in conjunction with FIG. 31 through 50, the optical subassembly 20 is shown in greater detail. The optical subassembly 20 includes a subassembly housing 52, a lens 54 and a lens cap 56. The housing 52 includes a (fiber adapter port) 60 that connects the fiberoptic cable 22 and holds it in position. The fiber adapter 62 accommodates the SMA905 connector 30. The lens 54 is held in place with a lens cap 56 and allows for the easy release of the lens for maintenance or replacement, without having to remove the main body 52. The optical subassembly 20 is made from either metal or a suitable polymer.

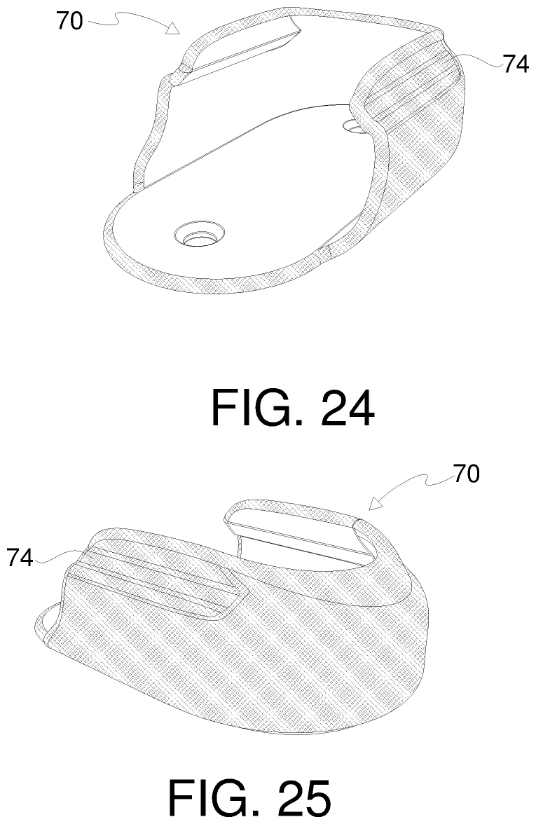

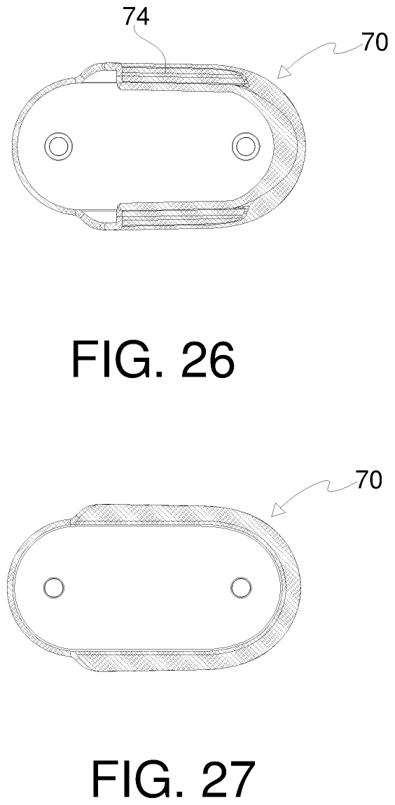

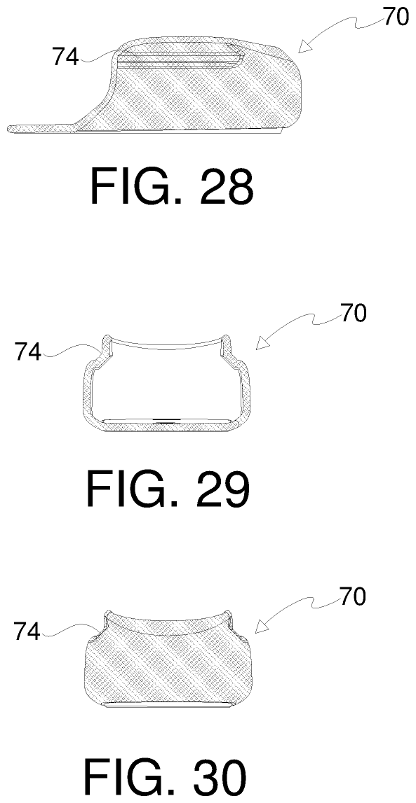

In conjunction with FIG. 24 through FIG. 30, a docking station 70 is shown for use in conjunction with the hand piece 10. The handle 14 of the hand piece 10 has two shaped profiles 72 on the lower portion of its handle, that coincide with shaped profiles 74 in the docking station 70. It is intended in a preferred design that the docking station 70 may be formed as a single piece, with a matching shaped profile 74 to the handle profile 72. The docking station may be designed to be fastened down to secure it, thereby creating a stable mount for the handpiece 10 when not in use. The docking station gets fastened down with either a double-sided adhesive tape, or a high tack hook and loop fastener tape or machine screws for a more permanent placement.

2. Operation of the Preferred Embodiment

In operation, the present invention provides a system and method for delivering laser therapy utilizing an improved ergonomic laser therapy hand piece device. The novel laser hand piece 10 is provided with the aim to improving the designs aesthetic, its reliability and its comfort. This is achieved with its right angle hand grip handle, along with its linear optic design, with the fiber cable exiting directly behind the lens of the hand piece. In the hand piece 10, the cable 22 exits the rear of the enclosure 18, running over the top of the user's hand thereby creating a downward force that is counter balanced by the right angle hand grip, thereby allowing for a more natural grip with finer control and comfort during use. The use of the linear optical subassembly 20 configuration means less parts are used, making it more reliable and less costly. The linear optic configuration means there are less connects between the laser light and the final lens, thereby ensuring a more stable beam, less prone to losing focus. Further, the robust optical subassembly 20 design allowing for ease of access to the lens 54, with a port 60 on the rear to accommodate the SMA fiber adapter, which in turn accommodates the SMA905 connection on the fiber cable. The spot size being emitted from the distal end of the hand piece is 40 mm at a distance of 10 mm from the surface being treated. The lens cap/cover is intended to provide easy access to the lens for cleaning and hygienic maintenance and is easily removed and replaced, being tightened with the user's fingers.

The docking station is a stand-alone part allowing the practitioner the freedom of placing the hand piece where it is most suitable for easy access during the treatments. The docking station has the option of being a mobile part that can be placed in multiple situations with the fiber cable length dictating the maximum extent for placement possible from the laser housing. The docking station is designed for easy insertion and extraction of the hand piece with the minimum amount of effort while still ensuring a secure seat for the laser hand piece when not in use.

In operation providing High Dose Laser Therapy (HDLT), the present invention may be used in many applications, including:
Anti inflammation. When targeted laser light causes vasodilatation, the lymphatic drainage system (drains swollen areas) is activated and a result is a reduction in swelling caused by bruising or inflammation and less pressure on pain receptors.
Anti Pain (analgesic). Targeted laser light facilitates better transfer of nerve impulses through the stimulation of neurotransmitter cells, reducing and eliminating pain through more effective functioning of the tissues surrounding the peripheral nerves and by temporary suppressing pain receptors.
Increased nerve regeneration and function. Slow recovery of nerve functions in damaged tissue can result in numbness and impaired limbs. The application of laser light speeds up the process of nerve cell reconnection and increases the amplitude of action potentials to optimize muscle action.
Improved vascular and lymphatic activity. Laser light will significantly increase blood flow to injured tissues as well as the formation of new capillary growth along with lymphatic activation.
Accelerated tissue repair and cell growth. Photons of laser light that penetrate deeply into tissue will accelerate cellular reproduction and tissue regeneration and growth. Laser light also activates stem cells in the treatment area, leading to faster wound healing and less scar tissue.
Trigger point resolution and acupuncture stimulation. Laser therapy stimulates muscle trigger points and acupuncture points on a noninvasive basis providing musculoskeletal pain relief.
Stem cell activation. Laser light has been documented to stimulate stem cell activation thus playing an active role in the tissue healing process.

The foregoing descriptions of specific embodiments of the present invention are presented for purposes of illustration and description. The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 15 U.S.C. §101, 102, or 103, nor should they be interpreted in such a way. Any unintended embracement of such subject matter is hereby disclaimed. They are not intended to be exhaustive nor to limit the invention to precise forms disclosed and, obviously, many modifications and variations are possible in light of the above teaching. The embodiments are chosen and described in order to best explain principles of the invention and its practical application, to thereby enable others skilled in the art to best utilize the invention and its various embodiments with various modifications as are suited to the particular use contemplated. It is intended that a scope of the invention be defined broadly by the Drawings and Specification appended hereto and to their equivalents. Therefore, the scope of the invention is in no way to be limited only by any adverse inference under the rulings of Warner-Jenkinson Company, v. Hilton Davis Chemical, 520 US 17 (1997) or Festo Corp. v. Shoketsu Kinzoku Kogyo Kabushiki Co., 535 U.S. 722 (2002), or other similar caselaw or subsequent precedent should not be made if any future claims are added or amended subsequent to this Provisional Patent Application.

Lasers can be dangerous if not properly used within the recommended safety parameters. Class IV lasers can burn a person's skin and ignite combustible materials. These dangers can arise from indirect or non-specular reflections of the beam, even off matte surfaces. Class 4 lasers are therefore rated as potential fire hazards and require specified safety prerequisites in the form of warning labels and proper use by trained professionals. There is a known need to ongoing improve the safety standards for high power class 4 laser handpieces.

The handpiece of the present disclosure may reduce this danger. While the handpiece can still burn a patient's skin if not used correctly, the inherent fire risk has been substantially reduced beyond that of laser therapy handpieces previously implemented due to the nature of the divergent lens provided herein. The lens provided herein continuously diverges the laser beam the further away from the handpiece that the beam travels, which it does at an angle of 91.01 degrees. This translates to an increase in spot size by more than 20 mm every 10 mm distance the beam is moved away from the handpiece. This reduces the likelihood of a fire inadvertently being started because of the immediate and continuous dissipation of the laser light energy the further away it travels from the laser therapy handpiece.

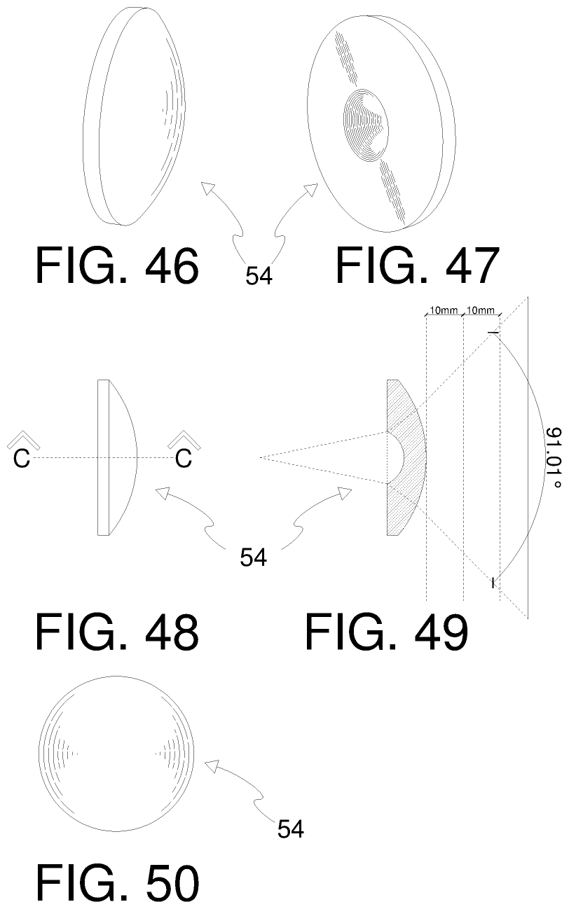

FIGS. 46 to 50 illustrate the divergent lens the TMA handpiece uses. The dashed line traveling through the lens in FIG. 49, illustrates the laser light beam's path. The vertical lines intersecting the dashed line of the beams path are measure lines to illustrate the divergence over the distance of 20 mm thereby illustrating the extreme nature of the divergence over a short distance.

From an occupational health and safety perspective, it is known that handheld tools should not have detailed depressions, profiles, ridges or valleys greater than 3 mm in depth on their surface in order not to cause pressure injury to soft tissue. Further the handle to the handheld device should have a shape that is smooth and in general conforms to the contours of a hand's grasp. The handle should be of a girth that ideally is suitable for both large and small hands and if a trigger (switch) is included that it sits comfortably aligned with the index finger. Thumb triggers should be avoided, and Ideally the handle should be suitable for both left and right-handed individuals.

There is a known need for improvements within the laser therapy field for handpiece comfort and handling. The handpiece provided herein complies with ergonomic criteria as specified by health and safety guidelines. The handpiece ensures a stress-free grip for the user and may prevent physiological or stress-related issues when the device is used for prolonged periods.

The handpiece handle provided herein is organically shaped to fit the human hand grip with the upper portion of the handle being slightly thinner than the base. The handle's trigger (switch) position facilitates the comfort of the index finger, thereby avoiding unwanted stress when holding the trigger for a prolonged period.

The base has a slight non-concentric bulge to better accommodate the human grip. The bulge simultaneously may strengthen the user's grip on the handle by providing a variance in the handle shape. The variance may allow for a mental distinction by the user of the handle shape from it top to its bottom. The mental distinction may allow for a proper grip adjustment for the user by promoting the user to adjust his/her hand up or down on the handle, or along its vertical axis.

The handle of the handpiece provided herein is smooth with a fine texture to facilitate a non-slip surface with no unwanted physical details such as the aforementioned depressions, profiles, ridges or valleys. The smoothness may promote comfort for the user, particularly over prolonged periods of use when stress ailments occur.

Secure placement of the laser therapy handpiece during treatment sessions is important. The laser therapy handpiece should be easily located during a treatment proximate the patient. Many known laser therapy handpieces have storage stations on the laser generator itself. Said storage stations tend to be profiled constructions that the handpiece rests in. That none or few of existing storage solutions secure the laser therapy handpieces means they can inadvertently be dislodged, causing damage to the laser handpiece or worse. The docking station of the present disclosure is a means of securing the handpiece when not in use, allowing for flexibility in its placement on any suitable surface, which may ideally be situated within the treatment area proximity.

The flexibility of the docking station's placement may allow for the user to provide a higher quality service. The user need not break his/her concentration on the patient to find the storage location for the handpiece. The docking station has two profiles that match the laser handpiece. This may allow for a seamless fit into the docking station that does not require hand-eye coordination typically required for such an exercise. This freedom may allow the user to maintain his/her concentration on the patient rather than on where he/she is placing the handpiece.

1. A medical laser for use in medical healing applications, comprising:
a hand piece having:
a main body containing an optical sub-assembly for delivering a dose of radiation; and
a hand-held grip angularly disposed from said main body, wherein said handheld grip has a vertical centerline that is approximately perpendicular to a horizontal centerline of the main body;
a high-powered class IV laser generator source in optical communication with said optical sub-assembly via a fiber optic cable; and
a docking station separate from and not connected to the laser generator, the docking station providing two shaped profiles accommodating two shaped profiles of the handheld grip.

2. The medical laser of claim 1, wherein said fiber optic cable is affixed at a proximal end to said laser generator and is affixed at a distal end to a rear location of said main body in a manner that provides a downward force that is counterbalanced by said hand held grip.

3. The medical laser of claim 2, wherein said optical sub-assembly is removably contained within said hand piece and comprises:
a subassembly housing supporting a lens;
a lens cap securing said lens to an end of said subassembly housing; and
a cable receiving channel at an opposite end of said subassembly housing, said cable receiving channel configured to connect said fiber optic cable to a fiber adapter.

4. The medical laser of claim 3, wherein the lens is divergent and continuously diverges a laser beam the further away from handpiece that the laser beam travels resulting in continuous dissipation of energy of the laser beam.

5. The medical laser of claim 1, where the hand-held grip is organically shaped with an upper portion of the grip slightly thinner than a base of the handle and the base having a non-concentric bulge.

6. The medical laser of claim 1, wherein the docking station is adapted to be positioned on a secure horizontal or vertical surface proximate a treatment area and away from the laser generator.

7. The medical laser of claim 2, wherein said proximal end of said fiber optic cable proximal to said laser generator terminates with separate connectors comprising an optical coupling including an SMA905 connector and an electrical coupling including a B series LEMO™ connector.

8. The medical laser of claim 7, wherein said SMA905 connector couples to a DIOWAVE™ class 4 laser's laser diode and said B series LEMO™ connector connects to a LEMO™ K series connection on said DIOWAVE™ laser.

9. The medical laser of claim 1, further comprising a control switch for activating and deactivating said laser generator.

10. The medical laser of claim 2, further comprising a control switch for activating and deactivating said laser generator.

11. The medical laser of claim 3, further comprising a control switch for activating and deactivating said laser generator.

12. The medical laser of claim 7, further comprising a control switch for activating and deactivating said laser generator.

13. The medical laser of claim 2, further comprising a strain relief assembly for said fiber optic cable at said second end.

14. A therapeutic medical laser system comprising:
a high-powered class IV laser generator source;
a fiber optic cable having a first end in optical communication with said laser generator and a second end opposite said first end;
a laser hand piece having:
a main body comprising:
an optical sub-assembly for delivering a dose of radiation;
an upper head having a centerline and containing the optical sub-assembly in optical communication with said second end, the second end physically attached to the upper head
a grip angularly dependent from said upper head and capable of orienting the upper head in a forward, vertical orientation relative to a user; and
a docking station separate from and not connected to the laser generator, the docking station providing two shaped profiles accommodating two shaped profiles of a handle of the laser hand piece.

15. The therapeutic medical laser system of claim 14, wherein said high powered class IV laser generator source comprises a Diowave™ Laser System provided by Technological Medical Advancements, Inc. of West Palm Beach, Fla. or its equivalent.

16. The therapeutic medical laser system of claim 14, wherein said centerline is generally horizontal; and
said grip forms a handle having a generally vertical centerline,
and wherein the vertical centerline is angularly disposed relative to the horizontal centerline at an approximately perpendicular angle.

17. The therapeutic medical laser system of claim 15, wherein said laser hand piece further comprises a head and a handle forming an enclosure consisting of two parts comprising:
a rear housing element; and
a front housing element mated to and fastened with said rear housing element.

18. The therapeutic medical laser system of claim 14, wherein the fiber optic cable is affixed at said second end at a position at said laser hand piece in a manner that provides a downward force that counter balances said laser hand piece about said hand held grip.

19. The therapeutic medical laser system of claim 14, further comprising a switch situated on a front of the grip for activating or deactivating the laser using an index finger.

20. The therapeutic medical laser system of claim 18, further comprising a switch situated on a front of the switch grip for activating or deactivating the laser using an index finger.
